(19) United States
(12) Patent Application Publication
(10) Pub. No.: US 2012/0107259 A1
(43) Pub. Date: May 3, 2012

(54) METHOD TO IMPROVE SKIN AND HAIR FRAGRANCE RETENTION FROM PERSONAL CARE COMPOSITIONS

(75) Inventors: Raymond Wei CEN, Hightstown, NJ (US); Binhua YANG, Pennington, NJ (US); Timothy Patrick KENNY, Flemington, NJ (US)

(73) Assignee: Elementis Specialties, Inc., Hightstown, NJ (US)

(21) Appl. No.: 13/286,814

(22) Filed: Nov. 1, 2011

Related U.S. Application Data
(60) Provisional application No. 61/408,748, filed on Nov. 1, 2010.

(51) Int. Cl. A61K 8/40; A61Q 15/00; A61K 8/44; A61Q 19/10; A61Q 1/00; A61K 8/42; A61K 8/45; A61Q 5/02; A61Q 5/12; A61Q 19/00

(52) U.S. Cl. 424/65; 424/70.28; 514/788; 510/123; 510/130

(57) ABSTRACT

A personal care composition and a method of using personal care compositions which results in increased fragrance retention when such compositions are applied to a keratin surface including hair and skin. The personal care compositions contain a quaternary ammonium compound, one or more fragrances, and a personal care acceptable carrier.

### RELATED APPLICATIONS

The present application claims priority to U.S. Provisional Patent Application No. 61/408,748, filed Nov. 1, 2010, the disclosure of which is incorporated by reference herein in its entirety.

### BACKGROUND

It is well known that mixtures of fragrance raw materials when deposited on hair or skin lose intensity and may change character with time, mainly due to factors such as poor sustantivity, wash loss, rub off, and differential evaporation. Many attempts have been made to minimize these drawbacks, but so far without notable success.

Personal Care products can be divided into two categories: leave-on and rinse-off. Many of them are indeed fragranced to bring pleasant aromas as an important consumer benefit. While leave-on products such as creams and lotions definitely have the challenge just described, rinse-off products such as shampoo, conditioner, and body wash face even more challenge(s) due to the loss of the main fragrance content during washing/cleansing processes. Therefore, as a key unmet consumer need, fragrance retention or prolongation has been intensively researched in personal care and cosmetic industries.

### SUMMARY

According to some embodiments, the present disclosure provides for a method of using personal care compositions containing quaternary ammonium compounds which result in increased fragrance retention when such compositions are applied to a keratin surface including hair and skin. The personal care compositions may be leave-on compositions or wash-off compositions.

In another embodiment, the present disclosure provides for a personal care composition including a quaternary ammonium compound, one or more fragrances and a personal care acceptable carrier.

In yet another embodiment, the present disclosure provides for the use of a quaternary ammonium compound to improve the fragrance retention of a personal care composition.

In some such embodiments, the quaternary ammonium compound corresponds to

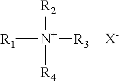

wherein: R₁ is independently selected from the group consisting of: C₁₀-C₂₂ alkyl group, C₁₀-C₂₂ ester group, R₅—C(═O)NH(CH₂)ₘ group wherein R₅ is C₁₀-C₂₂ alkyl where m ranges from 1 to 3 and mixtures thereof; R₂ and R₄ are each independently H, C₁-C₅ alkyl and polyoxyalkylene groups having polyoxyalkylene repeating units containing from 1 to 4 carbon atoms; R₃ is independently selected from the group consisting of R₁, R₂ and R₄; and X′ is an anion.

### BRIEF DESCRIPTION OF THE DRAWINGS

FIG. 1 is a graphical representation of a fragrance retention headspace analysis of two shampoo formulations.

FIG. 2 is a graphical representation of a fragrance retention headspace analysis of hair treated with two shampoo formulations.

FIG. 3 is a graphical representation of a fragrance retention sniff study analysis for two body wash formulations.

FIG. 4 is a graphical representation of an in-vivo fragrance retention forearm study for two body wash formulations.

FIG. 5 is a graphical representation of skin deposition of fragrance components for two body wash formulations.

FIG. 6 is a graphical representation of total skin deposition of fragrance components for four body wash formulations.

FIG. 7 is a graphical representation of skin deposition of fragrance components for two body wash formulations.

FIG. 8 is a graphical representation of skin deposition of fragrance components for two body wash formulations.

FIG. 9 is a graphical representation of a fragrance retention headspace analysis of two shampoo formulations.

### DETAILED DESCRIPTION

Previous efforts have been made to prolong the diffusion, as well as to improve other characteristics of fragrance materials, by e.g. increasing the fragrance raw material concentration or by using additives such as silicones, glycerol, polyethylene glycols and so on. Such additions, however, have never been adequate to increase the longevity of the fragrance odor. The present disclosure provides methods and compositions which meet this previously unmet need.

The present disclosure provides for personal care compositions and a method of using personal care compositions which result in increased fragrance retention when such compositions are applied to a keratin surface including hair and skin. The personal care compositions contain a quaternary ammonium compound, one or more fragrances; and a personal care acceptable carrier. In some embodiments, a personal care composition of the present invention includes a quaternary ammonium compound in an amount of about 0.1 wt % to about 10 wt %; about 0.2 wt % to about 9 wt %; about 0.3 wt % to about 8 wt %; about 0.4 wt % to about 7 wt %; about 0.5 wt % to about 6 wt %; about 0.6 wt % to about 5 wt %; about 0.7 wt % to about 4 wt %; about 0.8 wt % to about 3 wt %; about 0.9 wt % to about 2 wt %; 1 wt % to about 3 wt %; about 0.1 wt %; about 0.2 wt %; about 0.3 wt %; about 0.4 wt %; about 0.5 wt %; about 0.6 wt %; about 0.7 wt %; about 0.8 wt %; about 0.9 wt %; about 1 wt %; about 1 wt %; about 3 wt %; about 4 wt %; about 5 wt %; about 6 wt %; about 7 wt %; about 8 wt %; about 9 wt %; or about 10 wt %. In some embodiments, a personal care composition of the present invention include one or more fragrances in an amount of about 0.1 wt % to about 10 wt %; about 0.2 wt % to about 9 wt %; about 0.3 wt % to about 8 wt %; about 0.4 wt % to about 7 wt %; about 0.5 wt % to about 6 wt %; about 0.6 wt % to about 5 wt %; about 0.7 wt % to about 4 wt %; about 0.8 wt % to about 3 wt %; about 0.9 wt % to about 2 wt %; 1 wt % to about 3 wt %; about 0.1 wt %; about 0.2 wt %; about 0.3 wt %; about 0.4 wt %; about 0.5 wt %; about 0.6 wt %; about 0.7 wt %; about 0.8 wt %; about 0.9 wt %; about 1 wt %; about 1 wt %; about 3 wt %; about 4 wt %; about 5 wt %; about 6 wt %; about 7 wt %; about 8 wt %; about 9 wt %; or about 10 wt %.

In one embodiment, the increased fragrance retention, compared to a composition without a quaternary ammonium compound, corresponds to: at least 25%; at least 50%; at least 100%; at least 150%; at least 200%; at least 250%; at least 300%; at least 350%; at least 400%; at least 450%; at least 500%; at least 550%; at least 600%; at least 650%; at least 700%; at least 750%; at least 800%; at least 850%; at least 900%; at least 950%; at least 1000%; at least 1100%; at least 1200%; at least 1300%; at least 1400%; at least 1500%; at least 1600%; at least 1700%; at least 1800%; at least 1900%; at least 2000%; at least 2100%; at least 2200%; at least 2300%; at least 2400%; or at least 2500%. In another embodiment, the increased fragrance retention, compared to a composition without a quaternary ammonium compound, ranges from: 25% to 200%; 50% to 200%; 50% to 210%; 100% to 200%; 25% to 150%; 25% to 100%; 50% to 150%; 50% to 100%; 25% to 2500%; 25% to 2200%; 50% to 2200%; 650% to 2500%; 650% to 2200%; 700% to 2100%; 750% to 2000%; 800% to 1900%; 850% to 1800%; 900% to 1700%; 950% to 1600%; 1000% to 1500%; 1100% to 1400%; or 1200% to 1300%. In some embodiments, “fragrance retention” may be determined by a variety of methods including, but not limited to, direct analytical determination of initial deposition and residual levels of fragrance materials and their components; a headspace fragrance concentration analysis; an in-vivo sensory olfactory panel study. In some embodiments, such methods may be conducted in-vivo and/or in-vitro. Fragrance retention as determined by a total headspace analysis may be measured by the concentration of volatiles in a headspace vial containing a personal care composition after: 1 hour at room temperature; 18 hours at room temperature; and 18 hours at 50° C. Fragrance retention as determined by an analysis of fragrance-only concentration in head-space may be measured by the integrated concentration of selected fragrance components in a headspace vial containing a fragrance personal care composition or hair treated by such composition after, for example, 0 hours, 1 hour, 3 hours, and 6 hours. Fragrance retention as determined by a sniff study may be measured by the perceived olfactory scores of a test panel, such as on a scale of 1 to 10, at 0 hours, 1 hour, 3 hours, and 6 hours after each subject of the test panel washed their skin with the tested composition(s). Fragrance retention as determined by an in-vivo fragrance retention study may be measured by using an extraction cup to extract residual fragrance from the top layer of treated skin with medical isopropyl alcohol, at 0 hours, 1 hour, 3 hours, and 6 hours after the skin has been washed with the tested composition(s).

In one embodiment of the personal care composition, the quaternary ammonium compound may have formula 1:

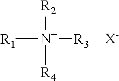

wherein: R₁ is independently selected from the group consisting of: C₁₀-C₂₂ alkyl group, C₁₀-C₂₂ ester group, R₅—C(═O)NH(CH₂)ₘ group wherein R₅ is C₁₀-C₂₂ alkyl where m ranges from 1 to 3 and mixtures thereof; R₂ and R₄ are each independently H, C₁-C₅ alkyl and polyoxyalkylene groups having polyoxyalkylene repeating units containing from 1 to 4 carbon atoms; R₃ is independently selected from the group consisting of R₁, R₂ and R₄; and X″ is an anion. The R₁ and optionally R₂ groups may be derived from naturally occurring fats or oils such as tallow, soybean oil, coconut oil, cottonseed oil, castor oil, safflower oil, meadowfoam seed oil, abyssinia oil and the like.

In some such embodiments, the polyoxyalkylene group may include: oxyethylene with the structure —(OCH2CH2); oxypropylene with the structure —(OCH(CH3)CH2— or —(OCH2(CH)CH3; oxytrimethylene with the structure —(OCH2CH2CH2)-; and 1,4-oxybutylene with the general structure —(OC4H8)-.

In such embodiments, physiologically acceptable counterions X which may, for example, be considered are halide ions, sulfate ions, phosphate ions, methosulfate ions and organic ions such as lactate, citrate, tartrate and acetate ions.

Exemplary quaternary ammonium cations, in accordance with embodiments of the present disclosure, may include one or more of the following: N-methyl-N,N,N tris (hydrogenated tallow) ammonium cation; N,N-dimethyl N,N, bis(hydrogenated tallow) ammonium cation; benzyl dimethyl hydrogenated tallow ammonium cation; N,N,N-trimethyl N-hydrogenated tallow ammonium cation; methyl benzyl bis(hydrogenated tallow) ammonium cation; dicocoylethyl hydroxyethylmonium cation; disoyoylethyl hydroxylethylmonium cation; distearoylethyl hydroxylethylmonium cation; N,N-bis(2-hydroxyethyl)-N-methyloctadecanaminium cation; N-coco alkyl N,N-bis(hydroxyethyl) N-methyl ammonium cation; N,N-Bis(2-hydroxyethyl)-N-methyloctadecanaminium; N-methyl-N-hydroxypropyl N,N-bis(hydrogenated tallow) ammonium cation; N-methyl-N-hydroxylethyl N,N-bis(hydrogenated tallow) ammonium cation; and distearoylpropyl trimonium cation.

Exemplary diquaternary ammonium cations, in accordance with embodiments of the present disclosure, may include one or more of the following: hydroxylpropyl bisoleyldimonium cation; hydroxypropylbisstearyldimonium cation; and hydroxypropyl bis(isostaramidopropyldimonium) cation.

In one embodiment, the quaternary ammonium cation is:

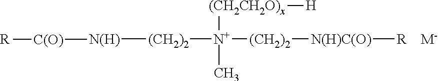

wherein R has the following composition: 6-65 wt. %-(CH₂)₃—CH═CH—(CH₂)₁₃—CH₃; 12-20 wt. % a mixture of —(CH₂)₃—CH═CH—(CH₂)₁₅—CH₃ and —(CH₂)₁₁—CH═CH—(CH₂)₇—CH₃; x is 2 and M⁻ is SO₄CH₃.

Fragrances include natural fragrance raw materials or synthetic fragrance raw materials (collectively referred to herein as “fragrance raw materials” or “FRM”) that can be characterized by their boiling point (B.P.), molar mass, and/or their octanol/water partitioning coefficient (P), otherwise known as logP and when calculated, known as ClogP and/or molecular weight and/or Kovats index.

The boiling points of many fragrance ingredients are given in, e.g., “Perfume and Flavor Chemicals (Aroma Chemicals),” Steffen Arctander, published by the author in 1969, and are incorporated herein by reference. In one embodiment, the fragrance ingredients useful in this invention may have boiling points of less than 250° C.

The octanol/water partitioning coefficient of a FRM is the ratio between its equilibrium concentrations in octanol and in water. In certain embodiments, the partitioning coefficients of the fragrance ingredients, useful in embodiments of this invention, will be referred to as the logarithm to the base 10, logP. In certain embodiments, the fragrance ingredients, useful in embodiments of this invention, may have logP in the range of: 1 to 8; 1 to 5; or 0.1 to 3. The personal care compositions of the present invention preferably comprise at least 0.1% of one or more fragrance ingredients.

The logP of many fragrance ingredients has been reported; for example, in the Pomona92 database, available from Daylight Chemical Information Systems, Inc. (Daylight CIS), Irvine, Calif. The logP values are also calculated by the “CLOGP” program, available from Daylight CIS.

KI values (Kovats Index) may be used to identify fragrance raw materials. The Kovats Retention Index system is an accurate method for reporting gas chromatographic data for interlaboratory substance identification. It is used for eliminating the effects of instrument parameters on retention correlations in peak identification by GC. In one embodiment, fragrance ingredients useful in the present invention have KI values: less than 1800: less than 1600; less than 1400. In one embodiment, fragrance ingredients useful in the present invention have KI values ranging from: 1800 to 1:1600 to 1; and 1400 to 1.

If it is to be perceptible, an odoriferous substance must be volatile, wherein, in addition to the nature of the functional groups and the structure of the chemical compound, an important role is also played by molar mass. Most odoriferous substances accordingly have molar masses of up to approx. 200 Dalton, while molar masses of 300 Dalton and above tend to be the exception.

Exemplary natural fragrance raw materials, which may be used alone or in combination with the present embodiments, include extracts of blossoms (lily, lavender, rose, jasmine, neroli, ylang-ylang), stems and leaves (geranium, patchouli, petitgrain), fruits (aniseed, coriander, caraway, juniper), fruit peels (bergamot, lemon, orange), roots (mace, angelica, celery, cardamom, costus, iris, calamus), woods (pine, sandalwood, guaiacwood, cedarwood, rosewood), herbs and grasses (tarragon, lemongrass, sage, thyme, chamomile), needles and branches (spruce, fir, pine, mountain pine), resins and balsams (galbanum, elemi, benzoin, myrrh, olibanum, opoponax).

Animal raw materials are also feasible, such as for example civet and castoreum.

Exemplary synthetic raw fragrance materials, which may be used alone or in combination with the present embodiments, include products of the ester, ether, aldehyde, ketone, alcohol and hydrocarbon types. Exemplary ester synthetic raw fragrance materials include benzyl acetate, phenoxyethyl isobutyrate, p-tert.-butylcyclohexyl acetate, linalyl acetate, dimethylbenzylcarbinyl acetate, phenylethyl acetate, linalyl benzoate, benzyl formate, ethylmethylphenyl glycinate, allylcyclohexyl propionate, styrallyl propionate, cyclohexyl salicylate, floramate, melusate, jasmecyclate and benzyl salicylate. Exemplary ether synthetic raw fragrance materials include benzyl ethyl ether and ambroxan. Exemplary aldehyde synthetic raw fragrance materials include linear alkanals with 8-18 carbon atoms, citral, citronellal, citronellyloxyacetaldehyde, cyclamen aldehyde, hydroxycitronellal, lilial and bourgeonal. Exemplary ketone synthetic raw fragrance materials include ionones, .alpha.-isomethylionone and methyl cedryl ketone. Exemplary alcohol synthetic raw fragrance materials include anethole, citronellol, eugenol, isoeugenol, geraniol, linalool, phenylethyl alcohol and terpineol. Exemplary hydrocarbon synthetic raw fragrance materials include terpenes and balsams such as limonene and pinene.

Further examples of fragrances which may be in the various embodiments as set forth herein are to be found for example in S. Arctander, Perfume and Flavor Materials, Vols. I and II, Montclair, N.J., 1969, self-publication or K. Bauer, D. Garbe und H. Surburg, Common Fragrance and Flavor Materials, 3rd ed., Wiley-VCH, Weinheim 1997.

In embodiments of the present disclosure, fragrance ingredients useful in the present invention are blended fragrances having materials corresponding to a top note material, a middle note material and a base note material. Synthetic fragrance raw materials may be blended to produce a blended fragrance composition which is often referred to as an accord. The components of such blended fragrances are categorized into three types based on their relative volatility; top, middle, and base notes. Top, middle, and base notes each serve a different purpose in the blending of fragrances. Based on volatility, these notes are described by those skilled in the art as: the base notes having the most long lasting aroma; the middle notes, have a medium volatility; and the top notes are the most volatile. Key to successfully formulating a fragrance-containing composition is the precise balance between these three groups of materials producing a fragrance-containing composition that diffuses during its evaporation in a manner which has an aesthetic quality.

It is recognized by those skilled in the art that descriptors which relate to aesthetic perceptions such as “top”, “middle” and “base” notes are relative terms. Synthetic fragrance raw materials categorized as a top note by one formulator usually has the identical classification among most other Perfumers. The same is true for the middle and base notes, however, occasionally one formulator may classify a given fragrance raw material as a middle note rather than a top note, or vice versa, but this fact does not diminish the utility of a given compound or its absolute identity.

In other embodiments of the present disclosure, fragrance ingredients useful in the present invention may be pro-fragrances or pro-accords which are stable in the personal care formulation but deliver a raw fragrance when applied to keratin surfaces. Pro-fragrances and/or pro-accords are chemically modified raw fragrance materials which deliver the raw fragrance when hydrolyzed. Suitable pro-fragrances can be formulated into the compositions of the present disclosure provided the product pH, carriers and adjunct materials are compatible with the pro-fragrance chemical form. Exemplary pro-fragrances and pro-accords are described in U.S. Pat. No. 7,018,978 incorporated by reference herein in its entirety.

The present disclosure provides for a method of providing enhanced fragrance retention in personal care compositions according to embodiments of the present invention including skin care products and hair care products. In one such embodiment, the personal care composition according to embodiments of the present invention is a leave-on product. In another embodiment, the personal care composition according to embodiments of the present invention is a wash-off product. In yet another embodiment, the personal care composition is a skin care product. In still yet another embodiment, the personal care composition is a hair care product. In some embodiments, the personal care composition according to embodiments of the present invention is suitable for use with natural fabrics, cotton, wood, silk, linen, or the like.

In some embodiments, suitable skin-care products may be independently selected from body washes, face wash, body oils, body lotions, body gels, treatment creams, skin protection ointments, moisturizing gels, moisturizing sprays, revitalizing body sprays, suntan lotions, cellulite gels and peeling preparations.

In some embodiments, suitable hair-care products may be independently selected from shampoos for humans and animals, in particular dogs and cats, hair conditioners, products for styling and treating hair, perming agents, hair relaxing agents, hair sprays and lacquers, hair gels, and hair fixatives.

The present personal care compositions or products can be in the form of creams, ointments, pastes, foams, gels, lotions, or sticks.

In one embodiment, the personal care composition according to various embodiments of the present invention includes a shampoo or body wash. Such compositions include various ingredients such as surfactants, cationic polymers, conditioning agents (hydrocarbon oils, fatty esters, silicones), anti-dandruff agents, suspending agents, viscosity modifiers, dyes, nonvolatile solvents or diluents (water soluble and insoluble), pearlescent aids, foam boosters, additional surfactants or nonionic cosurfactants, pediculocides, pH adjusting agents, perfumes, preservatives, chelants, proteins, skin active agents, sunscreens, UV absorbers, and vitamins.

Representative surfactants include anionic detersive surfactant, zwitterionic or amphoteric detersive surfactant, or a combination thereof. Suitable surfactants are described in U.S. Pat. No. 7,524,807 which is incorporated herein by reference in its entirety.

In another embodiment, the personal care composition according to various embodiments of the present invention includes hair conditioner composition. The hair conditioner composition may be a wash-off composition or leave-on composition. Representative ingredients include non-volatile silicone conditioning agents, humectants, moisturizer, thickening agents, amphoteric conditioning polymers, acidifiers, sequestrants, frizz control agents, or detanglers. A description of such ingredients is found in U.S. Pat. Nos. 6,709,648 and 5,759,527 each of which are incorporated herein by reference in their entirety.

The instant personal care compositions may further comprise, personal care acceptable ingredients such as fatty substances, organic solvents, oil structurants, surfactants, emulsifiers, thickeners, organic cationic deposition polymers, demulcents, opacifiers, additional colorants colorants, effect pigments, additional stabilizers, emollients, antifoaming agents, moisturizing agents, antioxidants, vitamins, peptides, amino acids, botanical extracts, particulates, perfumes, preservatives, polymers, fillers, sequestrants, propellants, alkalinizing or acidifying agents or other optional ingredients customarily formulated into cosmetics or other personal care compositions according to the invention.

### EXAMPLES

### Example 1

A headspace study was conducted to examine the fragrance retention of two shampoo formulations. Shampoo formulation B contained a diamidodiquaternary compound having the structure of:

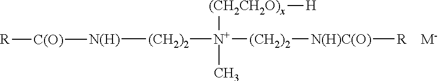

wherein R has the following composition: 6-65 wt. % —(CH₂)₃—CH═CH—(CH₂)₁₃—CH₃; 12-20 wt. % a mixture of —(CH₂)₃—CH═CH—(CH₂)₁₅—CH₃ and —(CH₂)₁₁—CH═CH—(CH₂)₇—CH₃; x is 2 and M⁻ is SO₄CH₃. The compound is commercially available from Elementis Specialties Inc. under the trade name Meadowquat® HG-70. The shampoo formulations (in wt %) are shown in the table below:

| | Formula A | Formula B |
|---|---|---|
| Ammonium Lauryl Sulfate | 12.36 | 12.35 |
| Alkyl Ether Sulfate | 4.12 | 4.12 |
| Fragrance Oil (Ocean Mist) | 3.00 | 3.00 |
| Meadowquat ® HG-70 | 0.00 | 3.00 |
| NaCl | 0.55 | 0.49 |
| Water | 79.96 | 77.04 |
| Total | 100.00 | 100.00 |

Shampoo Formula A was prepared mixing ammonium lauryl sulfate (“ALS”) and alkyl ether sulfate (“AES”) according to the formulation in the table above. The fragrance oil was added and stirred until a clear solution was obtained. The water was added with stirring. Trace amounts of NaCl were to adjust viscosity to form a shampoo texture while the shampoo went from clear to translucent.

Shampoo Formula B was prepared mixing ALS and AES according to the formulation in the table above. The fragrance oil was added stirred until a clear solution was obtained. The Meadowquat® HG-70 and water were added with stirring. Trace amounts of NaCl were to adjust viscosity to form a shampoo texture while the shampoo went from clear to translucent.

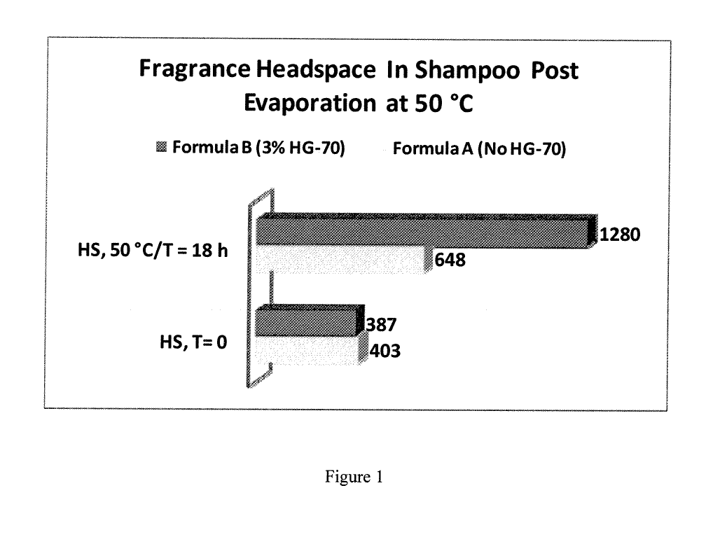

One (1) gram each of Shampoo Formulae A and B were placed in separate headspace vials. Then the two vials without caps were place in a 50° C. oven for 18 hours after which the vials were crimped then equilibrated for several hours. Two controls at zero hour were also included and processed similarly. The headspaces were then analyzed with GPC. The results are shown graphically in FIG. 1.

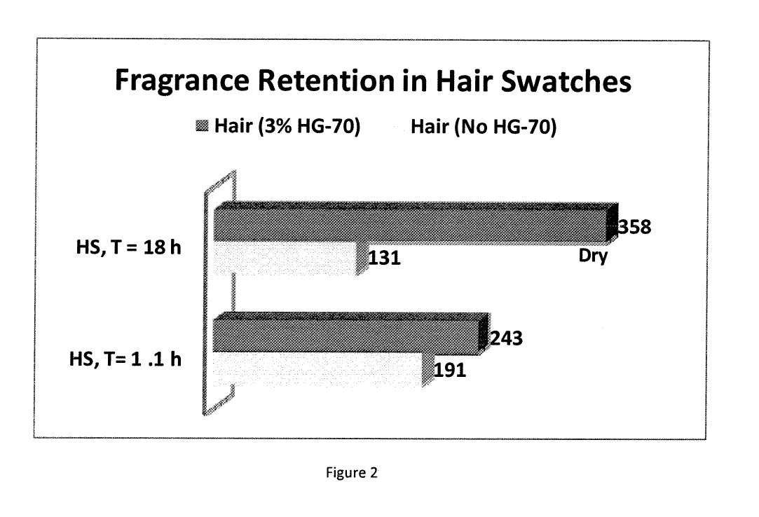

European triple damaged five (5) gram hair swatches were then washed with either Shampoo Formula A (3 swatches) or Shampoo Formula B (3 swatches). Each swatch was wet with three (3) grams of 40° C. water. The rinse water was set to 40° C. with a flow rate of 1 gallon per min. Five (5) grams of Shampoo Formula A were applied to a hair swatch followed by finger rubbing the swatch 40 times within a time period of two (2) minutes. The excess shampoo lather was removed followed by rinsing under running 40° C. water. Excess water was removed from the swatch to a final moisture level of three (3) grams water/swatch. The swatch was then placed in a ziploc bag to maintain the moisture level. The same procedure was followed for the three hair swatches washed with Shampoo Formula B. The six hair swatches were removed from the ziploc bag and air dried for 66 minutes while hanging from a bar. From each hair swatch, one (1) gram of hair was cut and placed in a five (5) mL headspace vial which was then crimped. A head space sample of each hair swatch was analyzed by gas chromatography (“GC”). The vial enclosed hair samples were dried for another 18 hours. The six (6) dried samples were equilibrated at room temperature followed by headspace analysis using GC. The cumulative peak area from the GC analysis are graphically shown in FIG. 2. The hair swatches washed with Shampoo Formula B, containing the Meadowquat® HG-70 showed a significant increase in the concentration of molecules in the vial headspace compared to the hair swatches washed with Shampoo Formula A. These results indicate that use of Meadowquat® HG-70 in a shampoo results in improved fragrance retention.

### Example 2

A forearm study was conducted using to test the fragrance retention of body wash formulations. Body Wash Formulae D and F contained a diamidodiquaternary compound having the structure of:

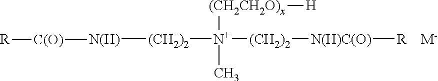

wherein R has the following composition: 6-65 wt. %-(CH₂)₃—CH═CH—(CH₂)₁₃—CH₃; 12-20 wt. % a mixture of —(CH₂)₃—CH═CH—(CH₂)₁₅—CH₃ and —(CH₂)₁₁—CH═CH—(CH₂)₇—CH₃; x is 2 and M⁻ is SO₄CH₃. The compound is commercially available from Elementis Specialties Inc. under the trade name Meadowquat® HG-70. The body wash formulations (in wt %) are shown in the table below:

| | Formula C | Formula D | Formula E | Formula F |
|---|---|---|---|---|
| | (1% Frag, | (1% Frag, | (3% Frag, | (3% Frag, |
| | 0% | 3% | 0% | 3% |
| Components | HG-70) | HG-70) | HG-70) | HG-70) |
| Ammonium lauryl sulfate | 47.0 | 47.0 | 47.0 | 47.0 |
| Alkyl ether sulfate | 17.0 | 17.0 | 17.0 | 17.0 |
| Fragrance Oil (Summer) | 1.00 | 1.00 | 3.00 | 3.00 |
| Meadowquat ® HG-70 | 0.00 | 3.00 | 0.00 | 3.00 |
| NaCl, 25% | 2.0 | 4.0 | 0.0 | 0.9 |
| Water | 33.0 | 28.0 | 33.0 | 29.1 |
| Total | 100.00 | 100.00 | 100.00 | 100.00 |

Forearm Sniff Study

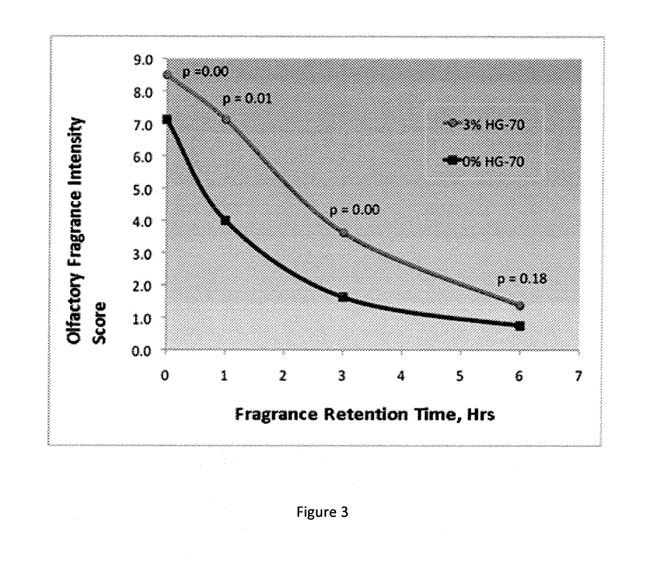

One (1) of eight (8) people each participated in the study. Each subject washed two forearms, one with Body Wash Formula C and the other with Body Wash Formula D. To conduct the forearm panel sniff study, the subjects then sniffed each forearm and evaluated the fragrance intensity by assigning olfactory scores on a scale of 1-10 for each, with 1 representing undetectable and 10 representing the strongest. Sniff tests were repeated every two (2) hours for eight (8) hours. Means and t-test were performed for each pair. The sniff study results are shown graphically in FIG. 3. The forearms washed with Body Wash Formula D, containing the Meadowquat® HG-70, showed significantly higher olfactory scores over the eight (8) hour evaporation time than the forearms washed with Body Wash Formula C. These results indicate that the use of Meadowquat® HG-70 in a body wash results in improved fragrance skin deposition and therefore retention.

In-Vivo Fragrance Retention

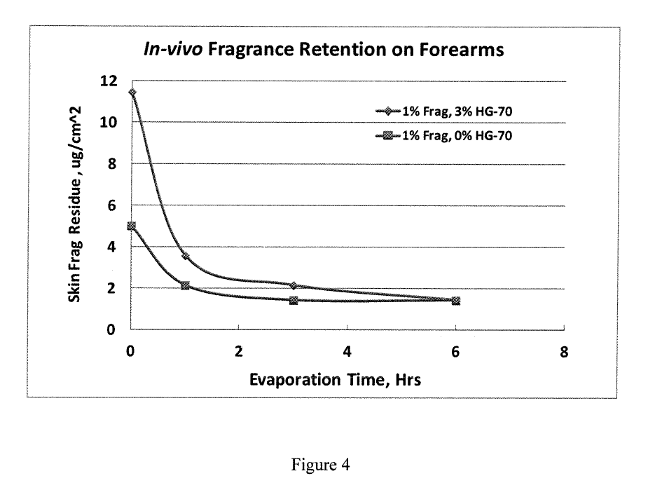

Subjects washed different areas of their skin with Body Wash Formula C and Body Wash Formula D. To conduct the in-vivo fragrance retention test, a four (4) cm diameter plastic extraction cup was used to extract residual fragrance from the stratum corneum (“SC”) layer of treated sites with medical isopropyl alcohol. Specifically, 1 ml of body wash and 2 ml of water were added and mixed in the cup area. A cotton swap was rubbed in the area for about one (1) minute. After a one (1) minute wait, the liquid was poured out and a pipette was used to remove the extra liquid accumulated along the cup's ring side. 9 ml of tap water was then added to the cup and mixed for 10 seconds before decanting the liquid and pipetting the extra liquid along the cup's side ring. Next, 9 ml of isopropyl alcohol was added to the cup for the initial deposition data. The area was rubbed with a cotton swab fifty (50) times, and then a clean pipette was used to remove the extraction liquid into a GC vial for analysis. The in-vivo fragrance retention test was performed at one (1) hour, three (3) hours, and six (6) hours. The in-vivo fragrance retention test results are shown graphically in FIG. 4. The skin washed with Body Wash Formula D, containing the Meadowquat® HG-70, showed significantly higher in-vivo fragrance retention over the six (6) hour evaporation time than the skin washed with Body Wash Formula C. These results also indicate that the use of Meadowquat® HG-70 in a body wash results in improved fragrance retention.

Total Skin Deposition

A skin deposition test was also conducted to assess the total fragrance deposition on the skin for Body Wash Formulae C, D, E, and F. Each subject marked 8 treatment sites on their forearms; 4 on right and 4 on left forearms. A plastic extraction cup of 4 cm diameter was used for each site. To each site was first added 1 mL of a body wash product followed by 2 mL of tap water. A cotton swab was used to rub the site 40 times. Treatment liquid with skin soil was removed by a pipette. 9 mL of fresh tap water was then added to the site for further rinse. The rinse liquid was removed completely by a pipette as well. At this time, time counting was started. Such practice was performed for all eight treatment sites in two forearms. Depending on time schedule, each site was extracted at a set time by 9 mL of medical isopropyl alcohol for fragrance residues at the time of 0, 1, 3, and 6 hours post treatment. GC analysis was used to determine analytical concentrations of fragrance in the IPA extracts. The data were then converted to skin fragrance depositions at the different time intervals for comparisons of time decay behavior and retention effect. The test results, based on GC analysis, are as follows:

| | Total Skin Fragrance Deposition, | | | | |
|---|---|---|---|---|---|
| | microgram/cm² skin | | | | |
| | 6.3 min | 8.0 min | 10.9 min | 12.9 min | Total area |
| Formula C | 1 | 1 | 3 | 4 | 9 |
| Formula D | 3.1 | 1.5 | 6.8 | 10.2 | 21.6 |
| Enhancement | 310% | 150% | 227% | 255% | 240% |
| Formula E | 2.3 | 0.7 | 0.9 | 7.1 | 8.7 |
| Formula F | 18.2 | 7.5 | 20.7 | 54.8 | 101.2 |
| Enhancement | 791% | 1071% | 2300% | 772% | 1163% |

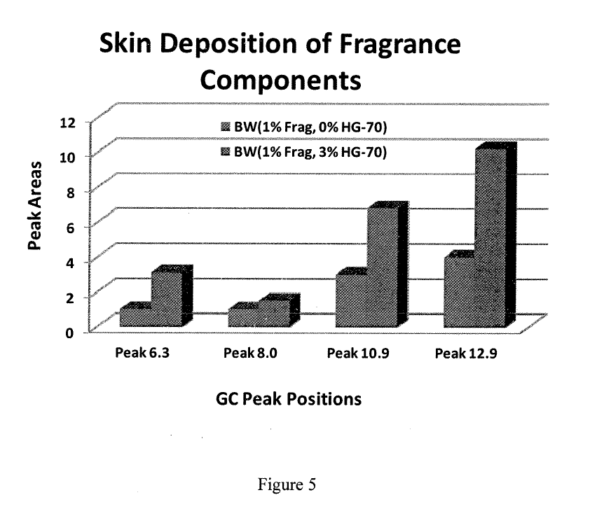

The skin deposition results for Formulae C and D are shown graphically in FIG. 5. The skin washed with Body Wash Formula D, containing the Meadowquat® HG-70, showed significantly higher skin deposition of fragrance components than Body Wash Formula C.

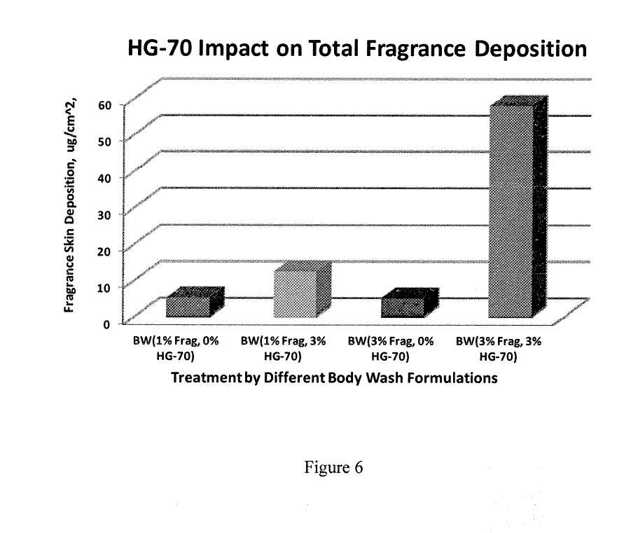

The total skin deposition results for Formulae C-F are shown graphically in FIG. 6. The skin washed with Body Wash Formulae D and F, containing the Meadowquat® HG-70, showed significantly higher total skin deposition than the skin washed with Body Wash Formulae C and E. These results also indicate that the use of Meadowquat® HG-70 in a body wash results in improved skin deposition.

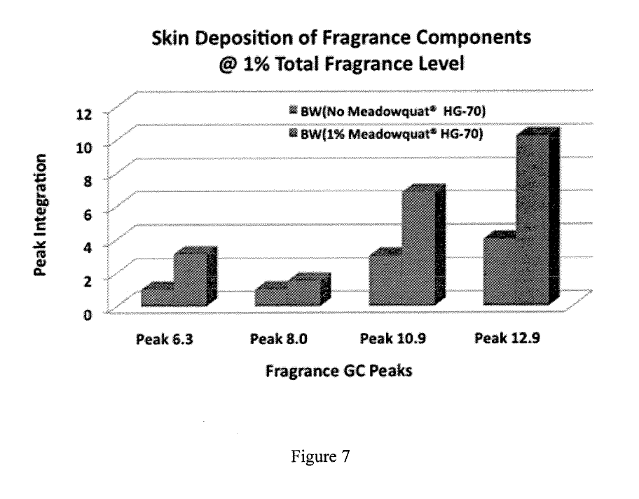

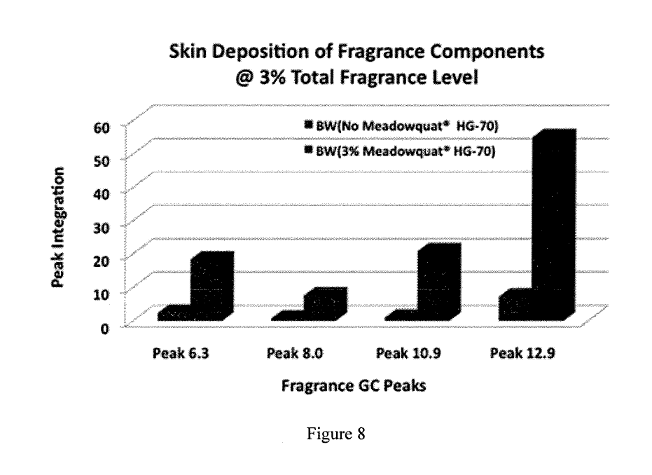

The cumulative peak area from the GC analysis are graphically shown in FIGS. 7 and 8. Again, the skin washed with Body Wash Formulae D and F, containing the Meadowquat® HG-70, showed significantly higher total skin deposition than the skin washed with Body Wash Formulae C and E. These results also indicate that the use of Meadowquat® HG-70 in a body wash results in improved skin deposition.

### Example 3

A hairswatch study was conducted to examine the fragrance retention of two shampoo formulations. Shampoo Formula H contained a diamidodiquaternary compound having the structure of:

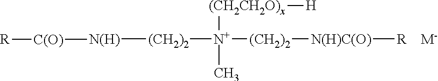

wherein R has the following composition: 6-65 wt. % —(CH₂)₃—CH═CH—(CH₂)₁₃—CH₃; 12-20 wt. % a mixture of —(CH₂)₃—CH═CH—(CH₂)₁₅—CH₃ and —(CH₂)₁₁—CH═CH—(CH₂)₇—CH₃; x is 2 and M⁻ is SO₄CH₃. The compound is commercially available from Elementis Specialties Inc. under the trade name Meadowquat® HG-70. The shampoo formulations (in wt %) are shown in the table below:

| | Formula G | Formula H |
|---|---|---|
| Components | (1% Frag, 0% HG-70) | (1% Frag, 3% HG-70) |
| Ammonium lauryl sulfate | 12.36 | 12.35 |
| Alkyl ether sulfates | 4.12 | 4.12 |
| Fragrance Oil | 1.00 | 1.00 |
| Meadowquat ® HG-70 | 0.00 | 3.00 |
| NaCl | 0.55 | 0.49 |
| Water | 81.97 | 79.04 |
| Total | 100.00 | 100.00 |

Anionic surfactants of the formulas were first mixed at room temperature with stirring. To the mixture was added an amount of Meadowquat® HG-70 according to the above formulation table. The required amount of fragrance from the above table was then added along with 80% of water. The mixture was stirred for 30 min to a homogeneous translucent or opaque liquid. The remaining water and the salt solution were then used to adjust the mixture to a finished body wash with the desired viscosity, such as the viscosity of Formula C.

The test results are as follows:

| | |
|---|---|
| | Fragrance in Headspace, |
| Dry Time, | micrograms/L |

| hrs | Formula G | Formula H |
|---|---|---|
| 0 | 442 | 426 |
| 1 | 262 | 493 |
| 3 | 188 | 153 |
| 6 | 116 | 99 |

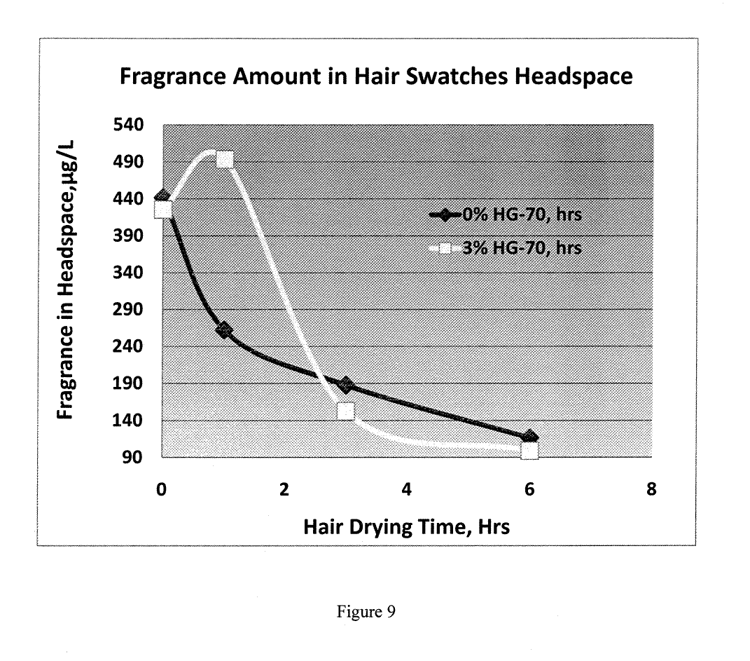

The test results are represented graphically in FIG. 9. The hair swatches washed with Shampoo Formula H, containing the Meadowquat® HG-70, showed a significant increase in the concentration of molecules in the vial headspace compared to the hair swatches washed with Shampoo G. These results indicate that use of Meadowquat® HG-70 in a shampoo results in improved fragrance retention.

The present disclosure may be embodied in other specific forms without departing from the spirit or essential attributes of the disclosure. Accordingly, reference should be made to the appended claims, rather than the foregoing specification, as indicating the scope of the disclosure. Although the foregoing description is directed to the preferred embodiments of the disclosure, it is noted that other variations and modification will be apparent to those skilled in the art, and may be made without departing from the spirit or scope of the disclosure.

What is claimed:

1. A method for providing enhanced fragrance retention to keratin fibers comprising the steps of:
applying to the keratin surface a personal care composition comprising:
a) a quaternary ammonium compound:
b) one or more fragrances; and
c) a personal care acceptable carrier.
wherein said keratin surface includes hair and skin; and
wherein said personal care composition provides improved fragrance retention to said keratin surface compared to a personal care composition without said quaternary ammonium compound.

2. The method of claim 1, wherein the quaternary ammonium compound has formula I: 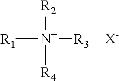
wherein:
R₁ is independently selected from the group consisting of: C₁₀-C₂₂ alkyl group, C₁₀-C₂₂ ester group, R₅—C(═O)NH(CH₂)ₘ group wherein R₅ is C₁₀-C₂₂ alkyl where m ranges from 1 to 3 and mixtures thereof;
R₂ and R₄ are each independently H, C₁-C₅ alkyl and polyoxyalkylene groups having polyoxyalkylene repeating units containing from 1 to 4 carbon atoms;
R₃ is independently selected from the group consisting of R₁, R₂ and R₄; and
X⁻ is an anion:

3. The method of claim 1, wherein the quaternary ammonium compound is independently selected from the group consisting of: N-methyl-N,N,N tris(hydrogenated tallow) ammonium cation; N,N-dimethyl N,N, bis(hydrogenated tallow) ammonium cation; benzyl dimethyl hydrogenated tallow ammonium cation; N,N,N-trimethyl N-hydrogenated tallow ammonium cation; methyl benzyl bis(hydrogenated tallow) ammonium cation; dicocoylethyl hydroxyethylmonium cation; disoyoylethyl hydroxylethylmonium cation; distearoylethyl hydroxylethylmonium cation; N,N-bis(2-hydroxyethyl)-N-methyloctadecanaminium cation; N-coco alkyl N,N-bis(hydroxyethyl) N-methyl ammonium cation; N,N-Bis(2-hydroxyethyl)-N-methyloctadecanaminium; N-methyl-N-hydroxypropyl N,N-bis(hydrogenated tallow) ammonium cation; N-methyl-N-hydroxylethyl N,N-bis(hydrogenated tallow) ammonium cation; distearoylpropyl trimonium cation and mixtures thereof.

4. The method of claim 2, wherein formula 1 corresponds to: 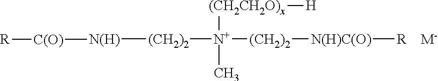
wherein R has the following composition: 6-65 wt. % —(CH₂)₃—CH═CH—(CH₂)₁₃—CH₃; 12-20 wt. % a mixture of —(CH₂)₃—CH═CH—(CH₂)₁₅—CH₃ and —(CH₂)₁₁—CH═CH—(CH₂)₇—CH₃; x is 2 and M⁻ is SO₄CH₃.

5. The method of claim 1, wherein the quaternary ammonium compound is independently selected from the group consisting of: hydroxylpropyl bisoleyldimonium cation; hydroxypropylbisstearyldimonium cation; hydroxypropyl bis(isostaramidopropyldimonium) cation and mixtures thereof.

6. The method of claim 1, wherein the personal care composition is independently selected from the group consisting of: a wash-off composition and a leave-on composition.

7. The method of claim 1, wherein the personal care composition is independently selected from the group consisting of: hair shampoo, hair conditioner, body wash, deodorant, anti-perspirant, body lotion, cosmetic, skin moisturizer and makeup.

8. A personal care composition comprising:
a. a quaternary ammonium compound having formula 1: 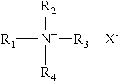
wherein:
R₁ is independently selected from the group consisting of: C₁₀-C₂₂ alkyl group, C₁₀-C₂₂ ester group, R₅—C(═O)NH(CH₂)ₘ group wherein R₅ is C₁₀-C₂₂ alkyl where m ranges from 1 to 3 and mixtures thereof;
R₂ and R₄ are each independently H, C₁-C₅ alkyl and polyoxyalkylene groups having polyoxyalkylene repeating units containing from 1 to 4 carbon atoms;
R₃ is independently selected from the group consisting of R₁, R₂ and R₄; and X⁻ is an anion;
b. one or more fragrances; and
c. a personal care acceptable carrier.

9. The composition of claim 8, wherein formula 1 corresponds to: 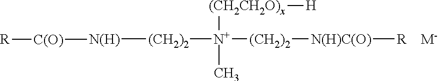
wherein R has the following composition: 6-65 wt. %-(CH₂)₃—CH═CH—(CH₂)₁₃—CH₃; 12-20 wt. % a mixture of —(CH₂)₃—CH═CH—(CH₂)₁₅—CH₃ and —(CH₂)_{H}—CH═CH—(CH₂)₇—CH₃; x is 2 and M⁻ is SO₄CH₃.

10. The composition of claim 8, wherein the quaternary ammonium compound is independently selected from the group consisting of: N-methyl-N,N,N tris(hydrogenated tallow) ammonium cation; N,N-dimethyl N,N, bis(hydrogenated tallow) ammonium cation; benzyl dimethyl hydrogenated tallow ammonium cation; N,N,N-trimethyl N-hydrogenated tallow ammonium cation; methyl benzyl bis(hydrogenated tallow) ammonium cation; dicocoylethyl hydroxyethylmonium cation; disoyoylethyl hydroxylethylmonium cation; distearoylethyl hydroxylethylmonium cation; N,N-bis(2-hydroxyethyl)-N-methyloctadecanaminium cation; N-coco alkyl N,N-bis(hydroxyethyl) N-methyl ammonium cation; N,N-Bis(2-hydroxyethyl)-N-methyloctadecanaminium; N-methyl-N-hydroxypropyl N,N-bis(hydrogenated tallow) ammonium cation; N-methyl-N-hydroxylethyl N,N-bis(hydrogenated tallow) ammonium cation; and distearoylpropyl trimonium cation.

11. The composition of claim 8, wherein the quaternary ammonium compound is independently selected from the group consisting of: hydroxylpropyl bisoleyldimonium cation; hydroxypropylbisstearyldimonium cation; hydroxypropyl bis(isostaramidopropyldimonium) cation and mixtures thereof.

12. The composition of claim 8, wherein the personal care composition is independently selected from the group consisting of: a wash-off composition and a leave-on composition.

13. The composition of claim 8, wherein the personal care composition is independently selected from the group consisting of: a shampoo, a hair conditioner and a body wash.
